(19) United States
(12) Patent Application Publication
(10) Pub. No.: US 2005/0178650 A1
(43) Pub. Date: Aug. 18, 2005

(54) Method for producing organic alkyne compounds

(75) Inventors: Robert Parker, Mannheim (DE); Robert Reinhard, Ludwigshafen (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: Basf Aktiengesellschaft, Ludwigshafen (DE)

(21) Appl. No.: 10/511,761

(22) PCT Filed: Apr. 25, 2003
(86) PCT No.: PCT/EP03/04288

(30) Foreign Application Priority Data
Apr. 29, 2002 (DE) 10219201.4

(51) Int. Cl. C07D 285/12; C07D 285/14; C07D 43/02; C07D 41/02

(52) U.S. Cl. 204/157.71; 204/157.72; 544/238; 544/296; 544/295; 548/136; 548/143; 546/255; 549/59; 568/686

(57) ABSTRACT

The invention relates to a method for producing organic alkyne compounds of formula (I), X—C═C—Y According to said method, organic halogen compounds of formula (Ia), X-Hal, are reacted with organic terminal alkyne compounds of formula (Ib), H—C═C—Y, X and Y representing the same or different organic radicals and Hal representing chlorine or bromine, in inert solvents under the action of microwave radiation, in the presence of at least one metallic compound and at least one base.

The present invention relates to a process for preparing organic alkyne compounds of the formula I
X—C≡C—Y (I)
by reacting organic halogen compounds of the formula Ia
X-Hal (Ia),
with organic terminal alkyne compounds of the formula Ib
H—C≡C—Y (Ib),
where X and Y are identical or different organic radicals and Hal is chlorine or bromine, in inert solvents under the action of microwave energy, in the presence of at least one metal compound and at least one base.

Under the customary conditions of the Sonogashira reaction, aryl or alkenyl halides are reacted with terminal alkyne compounds under palladium and copper salt catalysis at elevated temperature to give correspondingly substituted alkyne compounds.

A distinct reduction in the reaction time can be achieved by carrying out the reaction under the action of microwave radiation.

For instance, J.-X. Wang et al. (J. Chem. Research (S), 2000, p. 536-537) describe reactions of different terminal alkynes with organic iodine compounds in the presence of copper(I) iodide/triphenylphosphine and potassium carbonate in dimethylformamide (DMF). The comparison of the reactions show in table 2 of this publication, on the one hand under reflux of DMF, and on the other hand under the action of a microwave radiation source having an output of 375 W shows impressively that when comparable yields are obtained, the reactions in the latter case proceed more quickly than in the former case by factors of from 48 to 144.

Investigations of solvent-free reactions of aryl, heteroaryl and vinyl iodides with terminal alkynes in the presence of palladium/copper(I) iodide/triphenylphosphine and potassium fluoride supported on aluminum oxide under the action of microwave radiation have been carried out by G. W. Kabalka et al. (Tetrahedron Lett. 41, 2000, p. 5151-5154). The authors mention (p. 5152) that aryl chlorides and bromides did not react and that the starting materials were recovered unchanged.

We have now been found that, surprisingly, organic chlorine and bromine compounds can be reacted with terminal organic alkyne compounds to give alkyne derivatives in good to very good yields.

Accordingly, a process has been found for preparing organic alkyne compounds of the formula I
X—C≡C—Y (I)
by reacting organic halogen compounds of the formula Ia
X-Hal (Ia),
with organic terminal alkyne compounds of the formula Ib
H—C≡C—Y (Ib),
where X and Y are identical or different organic radicals in inert solvents under the action of microwave energy, in the presence of at least one metal compound and at least one base, wherein Hal is chlorine or bromine.

In this context, inert solvents are liquids or liquid mixtures which under the reaction conditions react neither with the reactants nor with the products.

In particular, such inert solvents are polar, aprotic liquids, since the use of protic liquids may lead to undesired secondary reactions which are triggered off by protonation.

To simplify the discussion, the terms “solvent” and “dissolve” will hereinbelow be used, even when in individual cases, for example, the base or bases or metal compound or metal compounds used are not completely dissolved, but are instead in suspension (or emulsion).

Preference is given to using those metal compounds which comprise a metal selected from the group consisting of magnesium, calcium, strontium, barium, titanium, zirconium, hafnium, iron, ruthenium, osmium, cobalt, rhodium, iridium, nickel, palladium, platinum, copper, silver, gold, zinc, cadmium and mercury. Particular preference is given to using copper compounds.

Emphasis is given to the metal halide compounds, in particular the chlorides and bromides, but also the iodides, of the metals mentioned. When these halides form adducts with triarylphosphines, for example triphenylphosphine, they are advantageously used in the form of these adducts.

Metal compounds further include the metals themselves, in particular the abovementioned metals in elemental form. Furthermore, combinations of more than one metal compound, more than one metal, and also combinations of metals and metal compounds may be used. The metal species which is catalytically active in the reaction does not necessarily have to be identical to the metal compounds added, but can instead only be formed in situ by reaction with the reactants and/or the base or bases.

The organic radicals X and Y are saturated or unsaturated hydrocarbon radicals, and also hydrocarbon radicals which contain both saturated and unsaturated moieties. The hydrocarbon radicals may further contain customary heteroatoms, such as nitrogen, oxygen, phosphorus, sulfur, fluorine, chlorine, bromine or iodine. The organic radicals X and Y customarily have molar masses of up to about 600 g/mol. However, in individual cases, the molar masses of the X and Y radicals may also be higher.

Preferred organic radicals X and Y contain saturated or unsaturated carbo- or heterocyclic radicals where both -Hal, i.e. chlorine or bromine, and H—C≡C— are bonded directly to the saturated or unsaturated carbo- or heterocyclic radicals.

In particular, X is a radical of the formula IIa
P¹—Y¹-(A¹-Y³)_{m′}-(T¹-B¹-)ₘ-T³- (IIa)
and
Y is a radical of the formula IIb
-T⁴-(B²-T²-)ₙ-(Y⁴-A²)_{n′}-Y²—P² (IIb)
where
P¹ and P² are each independently hydrogen, C₁-C₂-alkyl, a polymerizable group, a group suitable for polymerization or a radical which carries a polymerizable group or a group suitable for polymerization,
or
P¹ and/or P² each corresponds to a radical P^{1′} and/or P^{2′} which denotes a precursor group which is stable under the reaction conditions which can be reacted to give the corresponding polymerizable group or group suitable for polymerization P¹ and/or P² or the radicals P¹ and/or P² which carry a polymerizable group or a group suitable for polymerization,
Y¹, Y^{2,} Y³ and Y⁴ are each independently a single chemical bond, —O—, —S—, —CO—, —CO—O—, —O—CO—, —CO—N(R)—, —(R)N—CO—, —O—CO—O—, —O—CO—N(R)—, —(R)N—CO—O— or —(R)N—CO—N(R)—,
B¹ and B² are each independently a single chemical bond, —C≡C—, —O—, —S—, —CO—, —CO—O—, —O—CO—, —CO—N(R)—, —(R)N—CO—, —O—CO—O—, —O—CO—N(R)—, —(R)N—CO—O— or —(R)N—CO—N(R)—,
each R is, independently and irrespective of the meaning in each of Y¹ to Y⁴, B¹ and B², hydrogen or C₁-C₄-alkyl,
A¹ and A² are each independently spacers having from 1 to 30 carbon atoms,
T¹, T², T³ and T⁴ are each independently bivalent, saturated or unsaturated, carbo- or heterocyclic radicals and
m′, m, n′ and n are each independently 0 or 1.

The T¹ to T⁴ radicals in the formulae IIa and IIb are in particular those selected from the group consisting of

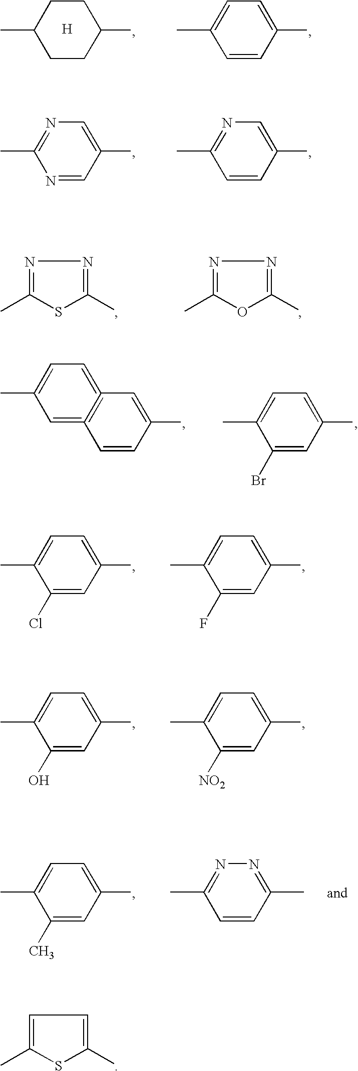

Useful C₁-C₁₂-alkyl radicals for P¹ and P² in formula I include branched and unbranched C₁-C₁₂-alkyl chains, for example methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-heptyl, n-octyl, 2-ethylhexyl, n-nonyl, n-decyl, n-undecyl and n-dodecyl.

Preferred P¹ and P² alkyl radicals are the branched and unbranched C₁-C₆-alkyl chains, such as methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl and n-hexyl.

Useful polymerizable groups or groups which are suitable for polymerization or radicals which carry a polymerizable group or a group suitable for polymerization (such groups or radicals are referred to hereinbelow simply as “reactive radicals”) for P¹ and P² are in particular: 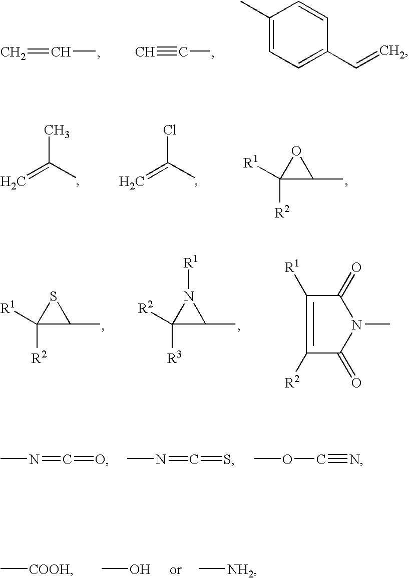
where the R¹ to R³ radicals can be identical or different and are each hydrogen or C₁-C₄-alkyl, such as methyl, ethyl, n-propyl, isopropyl, n-butyl, isobutyl, sec-butyl or tert-butyl.

Useful polymerizable groups for P¹ and P² are in particular the acrylate, methacrylate and vinyl radicals.

Useful C₁-C₄-alkyl radicals in the —CO—N(R)—, —(R)N—CO—, —O—CO—N(R)—, —(R)N—CO—O— and —(R)N—CO—N(R)— groups listed under the bridging units Y¹ to Y⁴, B¹ and B² include methyl, ethyl, n-propyl, isopropyl, n-butyl, isobutyl, sec-butyl and tert-butyl. When one or two R radicals are present in the Y¹ to Y⁴, B¹ and B² units, any R radicals present in the remaining units may be identical or different. The same applies to the case where there are two R radicals in one unit.

Useful spacers A¹ and A² include all groups known to those skilled in the art for this purpose. The spacers generally have from one to 30, preferably from one to 12, more preferably from one to six, carbon atoms and consist of predominantly linear aliphatic groups. They may be interrupted in the chain by, for example, nonneighboring oxygen or sulfur atoms or imino or alkylimino groups, for example methylimino groups. Useful substituents for the spacer chain include fluorine, chlorine, bromine, cyano, methyl and ethyl.

Examples of representative spacers include: 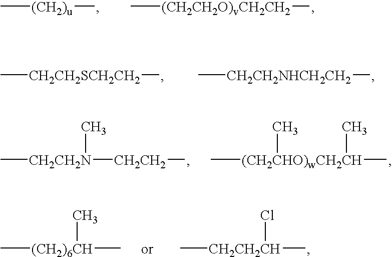
where u, v and w are integers and u is from 1 to 30, preferably from 1 to 12, v is from 1 to 14, preferably from 1 to 5, and w is from 1 to 9, preferably from 1 to 3.

Preferred spacers are ethylene, propylene, n-butylene, n-pentylene and n-hexylene.

The T¹ to T⁴ radicals are ring systems which may be substituted by fluorine, chlorine, bromine, cyano, hydroxyl, formyl, nitro, C₁ -C₂₀-alkyl, C₁ -C₂₀-alkoxy, C₁-C₂₀-alkoxycarbonyl, C₁ -C₂₀-monoalkylaminocarbonyl, C₁-C₂₀-alkylcarbonyl, C₁-C₂₀-alkylcarbonyloxy or C₁-C₂₀-alkylcarbonylamino.

Preferred T¹ to T⁴ radicals are:

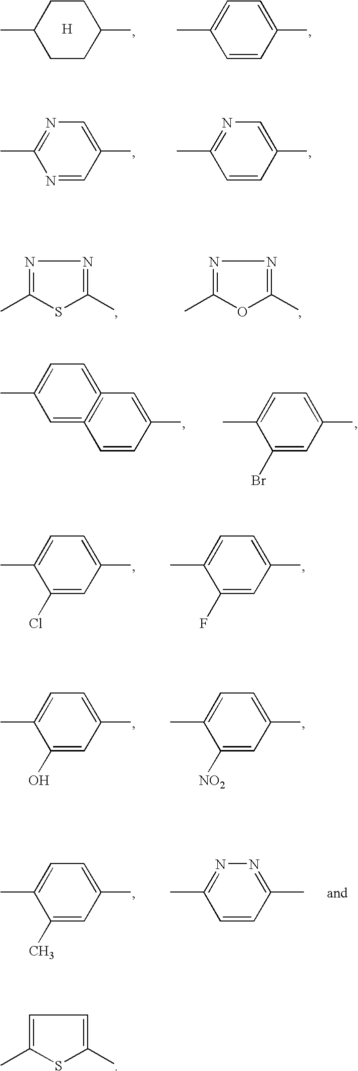

When the reactive P¹ and/or P² radicals are unstable under the reaction conditions, the reactants
P¹′—Y¹-(A¹-Y³)_{m′}-(T¹-B¹-)ₘ-T³-Hal and/or
H—C≡C-T⁴-(B²-T²-)ₙ(Y⁴-A²)_{n′}-Y²—P^{2′}
may be used as starting materials where the P¹′ and/or P²′ radicals are precursor groups which are stable under the reaction conditions and are converted to or substituted by the corresponding reactive P¹ and/or P² radicals in a subsequent step.

Compounds which, for example, have the construction
P¹′—Y¹-(A¹-Y³)_{m′}-(T¹-B¹-)ₘ-T³-C≡C-T⁴-(B²-T²-)ₙ-(Y⁴-A²)_{n′}-Y²—P^{2′}
may be regarded as direct products of the preparative process according to the invention.

Owing to retrosynthetic considerations, it may also be sensible to prepare the alkyne compounds by the process according to the invention which correspond to the fragments
-(A¹-Y³)_{m′}-(T¹B¹-)ₘ-T³-C≡C-T⁴-(B²-T²-)ₙ-(Y⁴-A²)_{n′}-Y²—P²,
-(A¹-Y³)_{m′}-(T¹-B¹-)ₘ-T³-C≡C-T⁴-(B²-T²-)ₙ(Y⁴-A²)_{n′}-Y²—P^{2′},
-(T¹-B¹-)ₘ-T³-C≡C-T⁴-(B²-T²-)ₙ-(Y⁴-A²)_{n′}-Y²—P²,
-(T¹-B¹-)ₘ-T³-C≡C-T⁴-(B²-T²-)ₙ-(Y⁴-A²)_{n′}-Y²—P^{2′},
P¹—Y¹-(A¹-Y³)_{m′}-(T¹-B¹-)ₘ-T³-C≡C-T⁴-(B²-T²-)ₙ-(Y⁴-A²)_{n′}-,
P¹′—Y¹-(A¹-Y³)_{m′}-(T¹-B¹-)ₘ-T³-C≡C-T⁴-(B²-T²-)ₙ-(Y⁴-A²)_{n′}-,
P¹—Y¹-(A¹-Y³)_{m′}-(T¹-B¹-)ₘ-T³-C≡C-T⁴-(B²-T²-)ₙ-,
P¹′—Y¹-(A¹-Y³)_{m′}-(T¹-B¹-)ₘ-T³-C≡C-T⁴-(B²-T²-)ₙ-,
-(A¹-Y³)_{m′}-(T¹-B¹-)ₘ-T³-C≡C-T⁴-(B²-T²-)ₙ-(Y⁴-A²)_{n′}-,
-(A¹-Y³)_{m′}-(T¹-B¹-)ₘ-T³-C≡C-T⁴-(B²-T²-)ₙ-,
-(T¹-B¹-)ₘ-T³-C≡C-T⁴-(B²-T²-)ₙ-(Y⁴-A²)_{n′}- or
-(T¹-B¹-)ₘ-T³-C≡C-T⁴-(B²-T²-)ₙ-
and then convert these in one or more subsequent steps using the appropriate complementary compounds to the target compounds
P¹—Y¹-(A¹-Y³)_{m′}-(T¹-B¹-)ₘ-T³-C≡C-T⁴-(B²-T²-)ₙ-(Y⁴-A²)_{n′}-Y²-P²′

Examples of compounds to which the above-listed fragments correspond include
HO-(A¹-Y³)_{m′}-(T¹-B¹-)ₘ-T³-C≡C-T⁴-(B²-T²-)ₙ-(Y⁴-A²)_{n′}-Y²—P²,
HO-(A¹-Y³)_{m′}-(T¹-B¹-)ₘ-T³-C≡C-T⁴-(B²-T²-)ₙ-(Y⁴-A²)_{n′}-Y²—P^{2′},
HO-(T¹-B¹-)ₘ-T³-C≡C-T⁴-(B²-T²-)ₙ-(Y⁴-A²)_{n′}-Y²—P²,
HO-(T¹-B¹-)ₘ-T³-C≡C-T⁴-(B²-T²-)ₙ-(Y⁴-A²)_{n′}-Y²—P^{2′},
P¹—Y¹-(A¹-Y³)_{m′}-(T¹-B¹-)ₘ-T³-C≡C-T⁴-(B²-T²-)ₙ-(Y⁴A²)_{n′}-OH,
P¹′—Y¹-(A¹-Y³)_{m′}-(T¹-B¹-)ₘ-T³-C≡C-T⁴-(B²-T²-)ₙ-(Y⁴-A²)_{n′}-OH,
P¹—Y¹-(A¹-Y³)_{m′}-(T¹-B¹)ₘ-T³-C≡C-T⁴-(B²-T²-)ₙ-OH,
P¹′—Y¹-(A¹-Y³)_{m′}-(T¹-B¹-)ₘ-T³-C≡C-T⁴-(B²-T²-)ₙ-OH,
HO-(A¹-Y³)_{m′}-(T¹-B¹-)ₘ-T³-C≡C-T⁴-(B²-T²-)ₙ-Y⁴-A²)_{n′}-OH,
HO-(A¹-Y³)_{m′}-(T¹-B¹-)ₘ-T³-C≡C-T⁴-(B²-T²-)ₙ-OH,
HO-(T¹-B¹-)ₘ-T³-C≡C-T⁴-(B²-T²-)ₙ-(Y⁴-A²)_{n′}-OH or
HO-(T¹-B¹-)ₘ-T³-C≡C-T⁴-(B²-T²-)ₙ-OH.

According to the definition of the X and Y radicals in the formulae IIa and IIb, the variables in the compounds listed are, in the same order in which they were listed, as follows:
P¹=hydrogen, Y¹═—O—,
P¹=hydrogen, Y¹═O—,
P¹=hydrogen, Y¹═—O—, m′=0.
P¹=hydrogen, Y¹═—O—, m′=0.
P²=hydrogen, Y²═—O—,
P²=hydrogen, Y²═—O—,
P²=hydrogen, Y²═—O—,n′=0,
P²=hydrogen, Y²═—O—,n′=0,
P¹═P²=hydrogen, Y¹═Y²═—O—,
P¹═P²=hydrogen, Y¹═Y²═—O—, n′=0,
P¹═P²=hydrogen, Y¹═Y²═—O—, m′=0 and
P¹═P²=hydrogen, Y¹═Y²═—O—, m′=n′=0.

Further, the hydroxyl group may be replaced by, for example, a carboxyl group (P¹=hydrogen and Y¹═—OCO— and/or P²=hydrogen and Y²═—COO—). In the difunctional compounds, both hydroxyl and carboxyl groups may also be present.

These hydroxyl or carboxylic acid or hydroxyl/carboxylic acid compounds which are given by way of example are again to be regarded as direct products of the preparative process according to the invention.

The reactants of the formulae Ia and Ib are customarily dissolved in a molar ratio of from 2:1 to 1:2 together with the at least one metal compound and the at least one base in the inert solvent. The solution is normally prepared at room temperature, but in individual cases, may also be prepared at higher or lower temperatures.

The temperature during the actual reaction under the action of microwave radiation is not critical. Customarily, the reaction is carried out at temperatures from room temperature to the boiling temperature of the solvent used.

Preference is given to using dimethylformamide (“DMF”), N-methylpyrrolidone (“NMP”) or a mixture of the two as solvent. Particular preference is given to using DMF as solvent (or as suspending medium) in the process according to the invention.

Preference is given to selecting the at least one base from the group consisting of alkali metal carbonates, alkali metal phosphates and tri(C₁-C₄-alkyl)amines, and emphasis is given to the alkali metal carbonates.

The group of suitable bases includes in particular sodium carbonate, potassium carbonate, sodium phosphate and potassium phosphate, trimethyl-, triethyl- and triisopropylamine.

Particular preference is given to using potassium carbonate.

In individual cases, the addition of potassium iodide may also be advantageous for the reaction. Whether there is such a positive effect and how much potassium iodide should optionally be added can be easily determined by preliminary experiments.

The output of the microwave radiation source is customarily from ten to hundreds of watts and should be selected according to the volume of the reaction batch. The correct power of the radiation source is customarily known to those skilled in the art and/or can be easily determined by preliminary experiments.

The alkyne compounds obtained are worked up and purified by customary organic synthesis methods.

### EXAMPLES

The Experiments Described Hereinbelow Use the Following:

| Substance | Source | Purity |
|---|---|---|
| 4-Chlorobenzoic acid | Acros | >99% |
| 4-Bromobenzoic acid | Merck | >99% |
| 4-Iodobenzoic acid | EMKA-Chemie | >99% |
| Phenylacetylene | Aldrich | >98% |
| Copper(I) iodide | Merck | >99% |
| Triphenylphosphine | Merck | >99% |
| Potassium carbonate | Merck | >99.9% |
| (ground) | | |
| Dimethylformamide | BASF | >99% |
| (“DMF”) | | |
| Potassium iodide | J. T. Baker | >99% |

Experimental Procedure:
General Reaction Equation:

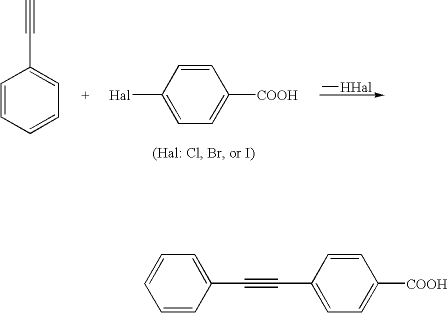

5 mmol of 4-halobenzoic acid (halo: chloro, bromo or iodo), 7.5 mmol of phenylacetylene, 0.5 mmol of copper(I) iodide, 1.0 mmol of triphenylphosphine, 7.5 mmol of potassium carbonate and 10 ml of DMF were initially charged under an argon atmosphere into a 100 ml four-neck flask provided with a magnetic stirrer, heated within 5 min to a temperature of 155° C. and subjected at reflux for 20 min to the maximum radiation output of a microwave device (MLS-Ethos 1600; unpulsed; magnetron frequency 2450 MHz; maximum output 375 W).

The workup was carried out by filtering off the solid (substantially in potassium carbonate), washing with 100 ml of dichloromethane and extracting the solution obtained three times with 50 ml each time of a saturated, aqueous sodium chloride solution. The dichloromethane solution was dried over sodium sulfate and then the solvent was removed on a rotary evaporator.

For comparative purposes, experiments were also carried out with the addition of 0.5 mmol of potassium iodide. The amounts of the remaining substances used were unchanged; the experimental procedure and workup were likewise identical to those described above.

Results:

The experimental results are reported in the following table.

| | | Potassium | 4-Halobenzoic |
|---|---|---|---|
| | Yield | iodide | acid |
| Example | (% of theory) | addition | Halo = |
| 1 | 33.0 | − | I |
| (Comparative) | | | |
| 2 | 74.4 | − | Cl |
| 3 | 56.5 | + | Cl |
| 4 | 54.5 | − | Br |
| 5 | 38.6 | + | Br |

When 4-iodobenzoic acid was used (example 1 (comparative), the lowest yields by far were obtained, but when 4-bromo- and in particular 4-chlorobenzoic acid were used (examples 4 and 5, and 2 and 3 respectively), distinctly higher yields of the desired target compound were obtained. In the experiments carried out here, the addition of potassium iodide (examples 3 and 5) caused a deterioration compared to the potassium iodide-free experimental procedure (examples 2 and 4). However, it is conceivable that, in individual cases, the addition of potassium iodide may have an advantageous effect.

1. A process for preparing organic alkyne compounds of the formula I
X—C≡—C—Y (I)
by reacting organic halogen compounds of the formula Ia
X-Hal (Ia),
with organic terminal alkyne compounds of the formula Ib
H—C≡—C—Y (Ib),
where X and Y are identical or different organic radicals in inert solvents under the action of microwave energy, in the presence of at least one metal compound and at least one base, wherein Hal is chlorine or bromine.

2. A process as claimed in claim 1 which is carried out in the presence of at least one metal compound selected from the group consisting of magnesium, calcium, strontium, barium, titanium, zirconium, hafnium, iron, ruthenium, osmium, cobalt, rhodium, iridium, nickel, palladium, platinum, copper, silver, gold, zinc, cadmium., a mercury and mixtures thereof.

3. A process as claimed in claim 1 which is carried out in the presence of a copper compound.

4. A process as claimed in claim 1, wherein X and Y are identical or different and are each organic radicals which contain saturated or unsaturated carbo- or heterocyclic radicals where both -Hal and H—C≡C— are bonded directly to said saturated or unsaturated carbo- or heterocyclic radicals.

5. A process as claimed in claim 1, wherein
X is a radical of the formula Ia
P¹—Y¹-(A¹-Y³)_{m′}-(T¹-B¹-)ₘ-T³- (IIa)
and
Y is a radical of the formula IIb
-T⁴-(B²-T²-)ₙ-(Y⁴-A²)_{n′}-Y²—P² (IIb)
where
P¹ and P² are each independently hydrogen, C₁-C₂-alkyl, a polymerizable group, a group suitable for polymerization or a radical which carries a polymerizable group or a group suitable for polymerization,
or
P¹ and/or P² each corresponds to a radical P^{1′} and/or P^{2′} which denotes a precursor group which is stable under the reaction conditions which can be reacted to give or be substituted by the corresponding polymerizable group or group suitable for polymerization P¹ and/or P² or the radicals P^{1′} and/or P^{2′} which carry a polymerizable group or a group suitable for polymerization,
Y¹, Y², Y³ and Y⁴ are each independently a single chemical bond, —O—, —S—, —CO—, —CO—O—, —O—CO—, —CO—N(R)—, —(R)N—CO—, —O—CO—O—, —O—CO—N(R)—, —(R)NCO—O— or
—(R)N—CO—N(R)—,
B¹ and B² are each independently a single chemical bond, —C≡C—, —O—, —S—, —CO—, —CO—O—, —O—CO—, —CO—N(R)—, —(R)N—CO—, —O—CO—O—, —O—CO—N(R)—, —(R)N—CO—O— or
—(R)—CO—N(R)—,
each R is, independently and irrespective of the meaning in each of Y¹ to Y⁴, B¹ and B², hydrogen or C₁-C₄-alkyl,
A¹ and A² are each independently spacers having from 1 to 30 carbon atoms,
T¹, T², T³ and T⁴ are each independently bivalent, saturated or unsaturated, carbo or heterocyclic radicals and
m′, m, n′ and n are each independently 0 or 1.

6. A process as claimed in claim 5, wherein the T¹ to T⁴ radicals in the formulae IIa and IIb are selected from the group consisting of 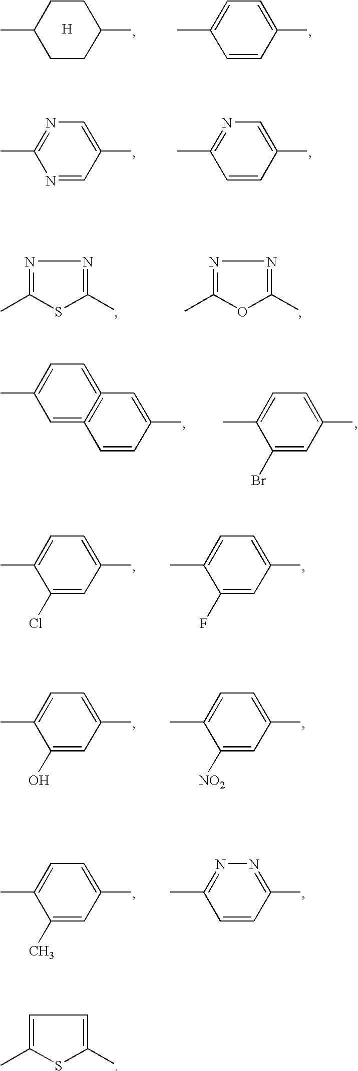
and mixtures thereof.

7. A process as claimed in claim 1, wherein the inert solvent used is dimethylformaniide or N-methyl-pyrrolidone or a mixture of the two.

8. A process as claimed in claim 1, wherein the inert solvent used is dimethylformamide.

9. A process as claimed in claim 1, wherein the at least one base is a compound selected from the group consisting of alkali metal carbonates, alkali metal phosphates, tri(C1-C4-alkyl)amines and mixtures thereof.

10. A process as claimed in claim 1, wherein the base used is at least one alkali metal carbonate.

11. A process as claimed in claim 1, wherein the base used is potassium carbonate.
